(19) United States
(12) Patent Application Publication
(10) Pub. No.: US 2010/0317763 A1
(43) Pub. Date: Dec. 16, 2010

(54) Denture Adhesive Articles

(76) Inventors: Jayanth Rajaiah, Loveland, OH (US); Franco Silva Medeiros, Loveland, OH (US); Luisa Navarro Cerda, Cincinnati, OH (US); Robert Scott Leonard, Fairfield, OH (US); Steven Daryl Smith, Fairfield, OH (US); Mark William Hamersky, Indian Springs, OH (US); Rafael Edmundo Bras, West Chester, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/542,461

(22) Filed: Aug. 17, 2009

Related U.S. Application Data
(63) Continuation-in-part of application No. 11/590,231, filed on Oct. 31, 2006, now abandoned.
Application No. 11/590,231 is a continuation-in-part of application No. 11/590,224, filed on Oct. 31, 2006, now abandoned.
Application No. 11/590,224 is a continuation-in-part of application No. 11/590,233, filed on Oct. 31, 2006, now abandoned.
Application No. 11/590,233 is a continuation-in-part of application No. 11/590,111, filed on Oct. 31, 2006, now abandoned.
Application No. 11/590,111 is a continuation-in-part of application No. 11/590,191, filed on Oct. 31, 2006, now abandoned.
Application No. 11/590,191 is a continuation-in-part of application No. 11/590,232, filed on Oct. 31, 2006, now abandoned.
Application No. 11/590,232 is a continuation-in-part of application No. 12/476,352, filed on Jun. 2, 2009.
Application No. 12/476,352 is a continuation-in-part of application No. 11/590,231, filed on Oct. 31, 2006, now abandoned.
(60) Provisional application No. 60/735,243, filed on Nov. 9, 2005.
(60) Provisional application No. 60/760,526, filed on Jan. 20, 2006.
(60) Provisional application No. 60/735,088, filed on Nov. 9, 2005.
(60) Provisional application No. 60/760,660, filed on Jan. 20, 2006.
(60) Provisional application No. 60/735,136, filed on Nov. 9, 2005.
(60) Provisional application No. 60/760,528, filed on Jan. 20, 2006.
(60) Provisional application No. 60/735,135, filed on Nov. 9, 2005.
(60) Provisional application No. 60/760,516, filed on Jan. 20, 2006.
(60) Provisional application No. 60/734,874, filed on Nov. 9, 2005.
(60) Provisional application No. 60/760,527, filed on Jan. 20, 2006.
(60) Provisional application No. 60/760,711, filed on Jan. 20, 2006.

(51) Int. Cl. A61K 6/00

(52) U.S. Cl. 523/120

(57) ABSTRACT

A denture adhesive article, comprising a denture adhesive component and a viscosity index improver, wherein the denture adhesive article is preformed.

### CROSS REFERENCE TO RELATED APPLICATION

This application is a continuation-in-part of U.S. application Ser. No. 11/590,231, filed Oct. 31, 2006, which claims the benefit of U.S. Provisional Application Nos. 60/735,243 filed Nov. 9, 2005; 60/760,526 filed Jan. 20, 2006; 60/735,088 filed Nov. 9, 2005; 60/760,660 filed Jan. 20, 2006; 60/735,136 filed Nov. 9, 2005; 60/760,528 filed Jan. 20, 2006; 60/735,135 filed Nov. 9, 2005; 60/760,516 filed Jan. 20, 2006; 60/734,874 filed Nov. 9, 2005 and 60/760,527 filed Jan. 20, 2006, and 60/760,711 filed Jan. 20, 2006 the substances of which are incorporated herein by reference.

This application is a continuation-in-part also of U.S. application Ser. No. 11/590,224, filed Oct. 31, 2006, the substance of which is incorporated herein by reference. This application is a continuation-in-part also of U.S. application Ser. No. 11/590,233, filed Oct. 31, 2006, the substance of which is incorporated herein by reference. This application is a continuation-in-part also of U.S. application Ser. No. 11/590,111, filed Oct. 31, 2006, the substance of which is incorporated herein by reference. This application is a continuation-in-part also of U.S. application Ser. No. 11/590,191, filed Oct. 31, 2006, the substance of which is incorporated herein by reference. This application is a continuation-in-part also of U.S. application Ser. No. 11/590,232, filed Oct. 31, 2006, the substance of which is incorporated herein by reference.

This application is a continuation-in-part also of U.S. application Ser. No. 12/476,352, filed Jun. 2, 2009, which claims priority to U.S. application Ser. No. 11/590,231, filed Oct. 31, 2006, the substances of which are incorporated herein by reference.

### TECHNICAL FIELD

This invention relates to denture adhesive articles and in particular to improved denture adhesive methods and articles which include an adhesive component and a viscosity index improver.

### BACKGROUND OF THE INVENTION

Ordinary removable dentures, dental plates and the like, comprise teeth mounted in a suitable plate or base. While dentures are traditionally fitted for the individual user, the fit can change over time which may result in slippage or discomfort. Whether the fit is good or bad, some users prefer extra security against slippage and/or dislodgement. Denture adhesives are used to temporarily adhere the dentures to the surfaces of the oral cavity, in particular the oral mucosa and give wearers the extra security they prefer. Denture adhesives are typically applied to the denture, oral surface, or both at the beginning of the day when the dentures are placed into the oral cavity. Unfortunately, denture adhesives tend to bioerode during the course of the day due to the action of saliva, chewing, drinking, and the like. This erosion leads to loss of adhesiveness, oozing of the adhesive into the oral cavity, dislodgement of the denture, food lodging under the denture, etc. As such, there is a need for improved denture adhesives.

### SUMMARY OF THE INVENTION

According to one embodiment, the present invention is directed to a denture adhesive article, comprising a denture adhesive component and a viscosity index improver, wherein the denture adhesive article is preformed.

In another embodiment, the present invention is directed to a denture adhesive article, comprising (a) from about 10.0% to about 60.0% of a denture adhesive component comprising AVE/MA, salts of AVE/MA, mixed salts of AVE/MA, or a combination thereof; and (b) from about 10% to about 90% of a viscosity index improver comprising microcrystalline wax, wherein the denture adhesive article is preformed.

These and other embodiments of the present invention will be more fully understood in light of the detailed description below.

### BRIEF DESCRIPTION OF THE DRAWINGS

While the specification concludes with claims particularly pointing out and distinctly claiming the invention, it is believed that the present invention will be better understood from the following description taken in conjunction with the accompanying drawings in which:

FIG. 1 is a top plain view of an embodiment of a concave shaped denture adhesive article having symmetrical dimensions;

FIG. 2 is a top plain view of an embodiment of a concave shaped denture adhesive article having asymmetrical dimensions.

### DETAILED DESCRIPTION OF THE INVENTION

A detailed description of embodiments of the present invention is given below.

### DEFINITIONS

The abbreviation “cm”, as used herein, means centimeter. The abbreviation “mm” as used herein, means millimeter. The abbreviation “g” as used herein, means gram. The abbreviation “P” as used herein, means Pascal. The abbreviation “s” as used herein means second. The abbreviation “Ps” as used herein means Pascal-second. The abbreviation “oz” as used herein, mean ounce.

The term “denture” as used herein refers to either the upper or lower denture, or both.

The term “viscosity index improver” as used herein refers to a material which makes the viscosity and/or rheology of a material into which it is incorporated more stable as its temperature is increased over a defined range. In the case of denture adhesive products, the defined range is between about 25° C. and about 60° C.

The term “denture adhesive article” and/or “article” as used herein refers to articles designed to fit, conform and adhere to contoured surfaces, such as a denture, as well as the gums or the roof of the mouth. The articles herein are substantially solid prior to use and can be picked up manually in substantially one piece and positioned on the denture. They are also preformed, that is, they are pre-shaped and ready to be applied.

The term “flexible” or “flexible article” as used herein means that a 0.67 mm thick piece of the article may be wrapped 180 degrees around a solid cylinder of 1 cm diameter without cracking upon visual observation.

The term “safe and effective adhesive amounts” as used herein means an amount sufficient to provide adherence to the oral cavity and/or provide adherence of a denture to the oral cavity, without toxicity to the user or damage to oral tissue.

By “safe and effective amount”, as used herein, is meant an amount of an agent high enough to significantly (positively) modify the condition to be treated or positively modify the benefit sought, but low enough to avoid serious side effects (at a reasonable benefit/risk ratio), within the scope of sound medical/dental judgment. The safe and effective amount of an agent may vary with the particular condition being treated, the age and physical condition of the patient being treated, the severity of the condition, the duration of treatment, the nature of concurrent therapy, the specific form of the source employed, and the particular vehicle from which the agent is applied.

The term “AVE/MA” as used herein refers to alkyl vinyl ether-maleic acid or anhydride copolymer. The term “mixed polymer salts” or “mixed salts”, as used herein, refers to salts of AVE/MA where at least 2 different cations are mixed on the same polymer with each other or with other salts.

The term “free acid” or “FA” component, as used herein, refers either to the unreacted carboxyl groups (—COOH) of AVE/MA copolymer plus any other monovalent cations of carboxyl groups (e.g., COONa) of the polymer. Monovalent cations include Group IA cations, such as sodium, potassium, hydrogen, etc. In one embodiment, the term “free acid” refers to the unreacted carboxyl groups (—COOH) of AVE/MA plus sodium and potassium cations. In another embodiment, the term “free acid” refers only to the unreacted carboxyl groups (—COOH) of the AVE/MA.

The term “toxicologically-acceptable”, as used herein, is used to describe materials that are suitable in their toxicity profile for administration to humans and/or animals.

The term “nonaqueous”, as used herein, means that the article is substantially free of added water. Substantially free means that no free water is added to the article, but the article may contain about 5% or less of water that comes in as part of other components.

The term “water-insoluble”, as used herein, refers to a material that, when exposed to water, does not dissolve, but may disperse to varying degrees. Generally, a material is water-insoluble if it is less than about 10% soluble in water.

The term “thermoplastic” as used herein refers to a material that melts, softens, becomes more flexible, extrudable, deformable, shapeable, moldable, flowable, processable, and/or changes rheology when exposed to heat. In one embodiment the material generally solidifies, hardens, and/or substantially returns to its original condition, when subsequently cooled.

The term “bioerodible”, as used herein, means that the article, when exposed to water or saliva, will erode over time due to physical and/or chemical action. The time necessary to erode the article can be any length of time from instantaneous to five days. The article may erode completely or substantially, however ultimately the article will lose its original form and/or integrity. For example, after application and use for at least about 24 hours in the oral cavity the article will not have sufficient product integrity to easily separate or peel, in its original form, from the denture or oral surface. In another embodiment some portion or residue from the article remains on the denture or oral surface after removing the denture from the oral cavity; however, this portion or residue from the article can be cleaned by brushing away with a toothbrush, but not easily separated from the denture.

The term “viscosity”, as used herein, refers to the internal resistance to flow or deformation of a material. This can be measured by the ratio of shearing stress to rate of shear; and in some embodiments where this ratio is not suitably measured, suitable rheological parameters such as loss modulus G″ or storage modulus G′ can be measured.

The percentages used herein to describe the cationic salt function of the alkyl vinyl ether-maleic acid or anhydride copolymers are defined as the stoichiometric percent of the total initial carboxyl groups reacted on the polymer.

Unless otherwise noted, the term “melting point” as used herein refers to the Drop Melting Point which is the temperature at which the material becomes sufficiently fluid to drop from the thermometer used in making the determination under prescribed conditions as listed in ASTM D-127. If ASTM D-127 is not suitable for the material in question, then ASTM D-3954 can be used instead.

Unless otherwise noted, the term “derivative” as used herein refers to when the primary polymeric backbone is left unchanged, but the side groups/chains and/or end groups are changed.

As used herein, the term “silicone” refers to siloxane polymers based on a structure of alternate silicon and oxygen atoms with various organic radicals attached to the silicon.

All other percentages used herein are by weight of the composition unless otherwise indicated.

All measurements referred to herein are made at 25° C. unless otherwise specified.

### Denture Adhesive Articles and Methods

Denture adhesives have become a daily product for many people who are looking for better fit and/or more security when wearing dentures. This has driven consumer demand for products which have improved properties like long-lasting hold, for example. The present denture adhesive articles deliver improvements on such desirable properties.

In general, the denture adhesive articles of the present invention include an adhesive component and a viscosity index improver. Historically, viscosity index improver was a term associated with the lubricant industry. The viscosity of a lubricant is closely related to its ability to reduce friction. The most desirable lubricant is one which will allow the easiest movement of two surfaces while still forcing the two moving surfaces apart, because this results in the lowest friction. However, as the viscosity of liquids tends to decrease as the temperature increases, many lubricants which work at lower temperatures are not thick enough to work at higher temperatures and those that are thick enough at the higher temperatures have a tendency to be too thick to work at the lower temperatures.

For example, the automotive industry requires lubricants which can perform across a wide range of conditions, like those found in an engine. Automotive lubricants must reduce friction between engine components when it is started from cold (relative to engine operating temperatures) as well as when it is running (up to 200° C.). The best oils (i.e. lubricants) will not vary much in viscosity over such a temperature range and therefore will perform well throughout.

In order to better predict the range of temperatures at which a lubricant would work, the Society of Automotive Engineers established the Viscosity Index. The Viscosity Index highlights how a lubricant's viscosity changes with variations in temperature. The Viscosity Index shows the viscosity of materials at an arbitrary “low” temperature of 100° Fahrenheit (40° C.) and an arbitrary “high” temperature of 210° F. (100° C.).

After understanding the properties of lubricants over the set temperature ranges, it was discovered that adding certain types of compounds to the lubricants would make the viscosity of the lubricants more consistent through a broader temperature range. Thus, there was less of a decrease in the viscosity of the lubricant at the higher temperatures. Having a higher viscosity at the higher temperature allowed the lubricants to work better at the higher temperatures. The materials added to increase the viscosity at higher temperatures were defined as viscosity index improvers.

It has surprisingly been discovered that application of that principle also has relevance to denture adhesives. In general, denture adhesive compositions comprise a denture adhesive component (salts of AVE/MA, for example) dispersed in a water insoluble component (petrolatum, for example). During use, the moisture in the saliva penetrates through the water insoluble component and hydrates the denture adhesive component. This makes the denture adhesive component sticky to the mucosal tissue and denture surface. The amount of hydration is influenced by the amount of denture adhesive component, the amount of water insoluble vehicle, and the viscosity of the water insoluble vehicle, all three of which contribute to the overall viscosity of the denture adhesive. The viscosity of the denture adhesive composition contributes to the rate and/or amount of hydration of the denture adhesive component. Over time, excess hydration due to excess saliva and/or liquids can lead to loss of some of the adhesive, thereby weakening it. As such, a denture adhesive composition that has a higher viscosity at mouth temperature due at least in part to the water insoluble vehicle would be more resistant to hydration. Simply put, the temperature-resistance of the viscosity imparted by the viscosity index improver results in resistance to excess hydration, which in turn results in more adhesive being retained over time-leading to extended and improved performance of the denture adhesive.

Thus, the use of viscosity index improvers alone or in combination with a water insoluble component will improve the hydration characteristics of a denture adhesive and thus provide an improved hold. The temperature range most relevant for denture adhesives is from room temperature (about 25° C.) which deals with the viscosity of the denture adhesive in the dispenser (outer package or inner dispensing package, for example) to about 40° C. which deals with the viscosity of the denture adhesive in the mouth. While the temperatures in the mouth can reach upward of 60° C. when drinking a hot beverage, looking at the behavior at 40° C. tends to be a good predictor of having increased beneficial properties at 60° C. as well. Thus, viscosity index improvers relevant for denture adhesives will make the viscosity more stable over the range of functional temperatures (i.e. about 25° C. to about 60° C.).

In light of the above, in one embodiment the denture adhesive article comprises a homogeneous mixture of the denture adhesive component and the viscosity index improver. In another embodiment, the denture adhesive article comprises a uniform mixture of the adhesive component dispersed within the viscosity index improver.

### Denture Adhesive Articles

The articles of the present invention have numerous benefits. In one embodiment the articles herein minimize or avoid the problem of premature sticking during application of the article to the denture. That is, with some prior art denture adhesive articles, before the article can be properly positioned over a target surface on the denture, inadvertent contact of the article with the denture may cause premature sticking at one or more locations on the denture. This may inhibit proper positioning of the article. Premature sticking may also cause contamination or degradation of the article prior to final positioning on the denture.

In one embodiment the term “dry tack” as used herein means that present articles exhibit minimal and/or no adhesive or cling properties in the dry state until activated by pressure applied by a user. In one embodiment this characteristic permits the present articles to be stored and dispensed in any desired mode without encountering the difficulties of premature clinging or adhering to themselves, and without the need for separate release sheets, liners, spacers, or the like. At the same time, in one embodiment when pressure activated at the desired location and at the desired time, the articles can, in the dry state, exhibit sufficient adhesive properties to form a bond to most plastic surfaces including a denture surface, this bond being sufficiently strong to survive handling of the denture without bond failure. Therefore, in one embodiment the articles herein, in the dry state, adhere to a target denture surface only when pressed thereagainst, thereby minimizing or avoiding this problem of inadvertent adherence during positioning on the denture surface. In one embodiment then, the articles herein do not have to be moistened or wet prior to application to the denture, thus providing a simple and easy way to apply an article to the denture.

In one embodiment the term “dry tack” as used herein means that present articles exhibit minimal and/or no adhesive or cling properties until activated by pressure applied by the user after the article has been warmed by the hands of the user, potentially during the course of application of the article to the denture surface.

In another embodiment the articles herein are non-tacky to the touch prior to application to the denture.

In another embodiment the term “dry tack” as used herein means articles herein in a dry and un-wetted state, are capable of immediate bonding by surface attachment to a dry plastic, polymethyl methacrylate, and/or other denture prosthesis substrate, upon subjecting the article to pressure. In one embodiment the dry article, develops bonding by surface attachment to a dry denture prosthesis substrate upon the application of finger pressure whereby the article remains bonded under its own weight, and the article herein will not remain bonded to this dry substrate under its own weight without using finger pressure to apply the article to the substrate. In one embodiment the force or pressure may be generated by one or more fingers. This force or finger pressure, in one embodiment, may be applied for 1-10 seconds or longer. In another embodiment the bonding of the article to the substrate is maintained from about 10 seconds to about 3 minutes or longer, in another embodiment from about 30 seconds to about 1 minute or longer.

In one embodiment the dry tack of the article is from about 0.025, 0.1, 1, 10, 100, 1000 gram force/square centimeter to about 10, 100, 1000, 10,000, 50,000, 100,000 gram force/square centimeter and any combination thereof.

In one embodiment the dry tack of an article that can be repositioned is from about 0.025 grams/force square centimeter to about 0.30 grams/force square centimeter, and in another embodiment from about 0.025 gram force/square centimeter to about 0.25 gram force/square centimeter.

It is reported that the room temperature modulus of any tacky adhesive is less than 3×10⁶ dynes/cm² when measured at a frequency of 1 Hz. This finding is a criterion for tack and has been given the name “Dahlquist criterion for tack.” (Adhesion and Adhesives Technology, by Alphonsus Pocius, 2^{nd} Edition, 2002 Carl Hanser Verlag, Munich).

In one embodiment of the current invention, the article has a modulus greater than the ‘Dahlquist criterion for tack’ of about 3×10⁶ dynes/cm². In another embodiment, the article has a shear storage modulus G′ (measured in dynes/cm² at a frequency of about 1 Hz at about 25 C) greater than about 5×10⁶; in another embodiment greater than about 1×10⁷; in another embodiment greater than about 5×10⁷; and in another embodiment greater than about 8×10⁷.

In one embodiment the article has a shear storage modulus G′ (measured in dynes/cm² at a frequency of about 1 Hz at about 25 C) from about 1×10⁶, 3×10⁶, 5×10⁶, 1×10⁷, 5×10⁷, and 8×10⁷ to about 5×10⁸, 5×10⁷, 1×10⁸, 5×10⁹, 1×10⁹, and 1×10¹⁰ and/or any combination thereof.

In one embodiment the article has a flexural stiffness of less than about 10 grams/cm, in another embodiment less that about 5 grams/cm, in another embodiment less that about 3 grams/cm, in another embodiment less than about 2 grams/cm and in yet another embodiment from about 0.1, 0.5, 1, to about 2, 3, 5, 10 grams/cm, in any combination, flexural stiffness as measured on a Handle-O-Meter, model #211-300, available from Thwing-Albert Instrument Company of Philadelphia, Pa. as per test method ASTM D2923-95.

In one embodiment the articles herein have a normalized dislodgement force of from about 1100 to about 12,000 grams per sq.cm, in another embodiment from about 1300 to about 10,000 grams per sq.cm, in another embodiment from about 1200 to about 5000 grams per sq.cm, in another embodiment from about 1400 to about 5000 grams per sq.cm, in another embodiment from about 1300 to about 2500 grams per sq.cm, in another embodiment from about 1750 to about 2500 grams per sq.com. In another embodiment, the normalized dislodgement force is from about 1100, about 1200, about 1300, about 1400, about 1500, about 1750 grams per sq.cm. to about 12,000, about 10,000, about 7500, about 5000, about 2500, about 2250 grams per sq.cm, and/or any combination thereof. In one embodiment the dislodgement force ratio is from about 1.1, 1.2, 1.3, 1.4, 1.5, 1.6, 1.7, 1.8, 1.9, 2.0 to about 1.3, 1.4, 1.5, 1.6, 1.7, 1.8, 1.9, 2.0, 3, 4, 5, 6, 8, 10, and/or any combination thereof. In one embodiment the dislodgement force ratio is from about 1.1 to about 10, from about 1.1 to about 8, from about 1.3 to about 4, and/or from about 1.3 to about 2.5.

The articles herein are preformed, meaning they are pre-shaped, and ready to use. The articles are substantially solid prior to use, and can be picked up manually, in substantially one piece, and positioned on the denture. The denture adhesive articles may be in the form of a strip contained within a package. In one embodiment the articles are capable of being picked up manually, and positioned on the denture, resulting in little or no residue on the fingers. In another embodiment the articles comprise a single layer. In yet another embodiment the articles are laminates, composites, and/or comprise two or more layers.

The denture adhesive articles may also be identified by the ooze method (as defined herein). A denture adhesive article may have a normalized ooze amount of from about 0, 3, 5, 10, 15, 20, 25% of the total article to about 30, 25, 20, 15, 10, 5, 3% of the total article and/or any combination thereof and/or the ooze ratio is from about 0, 00001, 0.001, 0.01, 0.1, 0.2, 0.25, 0.3, to about 0.1, 0.2, 0.25, 0.3, 0.4, 0.5 and/or any combination thereof.

In one embodiment the article herein comprises ingredients of natural origin.

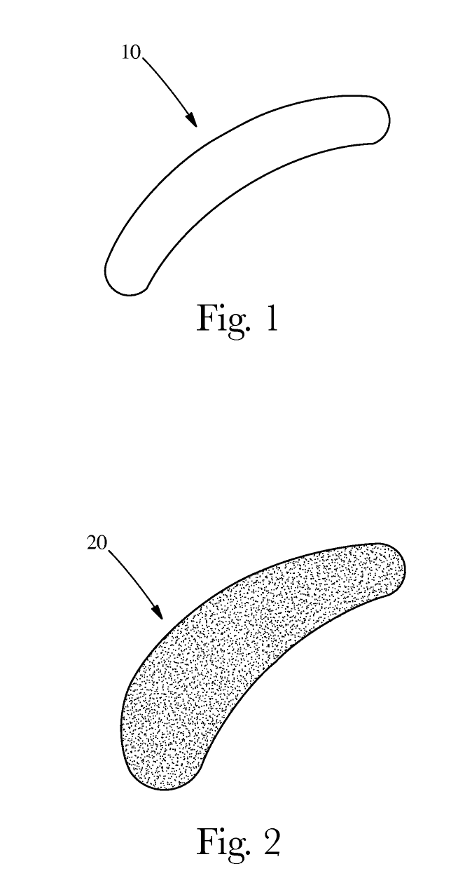

The denture adhesive article can have a variety of shapes and sizes including but not limited to a concave shape which is either symmetrical or asymmetrical. FIG. 1 shows a symmetrical concave shaped denture adhesive article 10. FIG. 2 shows an asymmetrical concave shaped denture adhesive article 20, in particular FIG. 2 shows a kidney shaped denture adhesive article 20.

In some embodiments the articles of the present invention are bio-erodible, non-aqueous, flexible, and/or thermoplastic. In some embodiments if the article contains polyethylene oxide, then the viscosity index improver and/or the optional water insoluble component is thermoplastic. In some embodiments if the article contains polyethylene oxide, the article may not include a fibrous paper web or paper laminate. In some embodiments, the article is substantially free of polyethylene oxide. In one embodiment the article herein is substantially free of honey mixed with alcohol. In another embodiment the article is substantially free of polyvinyl acetate resin in ethyl alcohol. In some embodiments the article is substantially free of polyvinyl acetate.

### Denture Adhesive Component

The present invention comprises a safe and effective adhesive amount of a denture adhesive component, generally at a level of from about 5% to about 99% by weight of the denture adhesive article. In other embodiments, the denture adhesive component is in the range from about 5%, 10%, 15%, 20%, 25%, 30%, 35%, 40% to about 20%, 30%, 40%, 50%, 60%, or any combination thereof. In other embodiments, the articles of the present invention comprise from at least 20 percent, by weight, and in another embodiment at least 30 percent, by weight, of the article, of a denture adhesive component.

In general, denture adhesive components are hydrophilic particles that become sticky when activated by moisture or are hydrophilic liquids. For those that activate with moisture, moisture can be present, for example, in the denture adhesive composition itself as well as in the oral cavity of the user. In varying embodiments, the denture adhesive components herein are mucoadhesive, hydrophilic, water soluble, have the property of swelling upon exposure to moisture, form a mucilaginous mass when combined with moisture, or any combination thereof. In a further embodiment the denture adhesive component is selected from the group consisting of: glycerin, poloxamer, Sorbitol, polyox, carbomer, polyacrylamides, poly peptides, natural gums; synthetic polymeric gums; AVE/MA; AVE/MA/IB; copolymers of maleic acid or anhydride and ethylene, styrene, and/or isobutylene, polyacrylic acid and/or polyacrylates thereof; polyitaconic acid, mucoadhesive polymers; water-soluble hydrophilic colloids; saccharide; cellulose; their derivatives, and combinations thereof. Examples of such materials include karaya gum, guar gum, gelatin, algin, sodium alginate, tragacanth, chitosan, acrylamide polymers, carboxypolymethylene, polyvinyl alcohol, polyamines, polyquarternary compounds, polyvinylpyrrolidone, polyvinylpyrrolidone copolymers, cationic polyacrylamide polymers, salts and mixed salts of AVE/MA, salts and mixed salts of AVE/MA/IB, salts and mixed salts of AVE/MA/Styrene, salts and mixed salts of AVE/MA/Ethylene; polymeric acids, polymeric salts, and copolymers thereof; polyitaconic acid salts, polyhydroxy compounds, their derivatives, and combinations thereof.

In one embodiment the denture adhesive component is selected from the group consisting of salts of AVE/MA, mixed salts of AVE/MA, cellulose derivatives (such as methylcellulose, carboxymethylcellulose, hydroxyethylcellulose, hydroxypropylcellulose, hydroxy-propylmethylcellulose, corn starch, and combinations thereof), polyethylene glycol, karaya gum, sodium alginate, chitosan, and combinations thereof. In yet another embodiment, the adhesive component is selected from the group consisting of mixed salts of AVE/MA, cellulose derivatives, and combinations thereof.

In another embodiment, the denture adhesive component is selected from the group consisting of: cellulose, cellulose derivatives, starch, starch derivatives, saccharide, saccharide derivatives, polyethylene oxides, polyethylene glycols, polyvinyl alcohols, carrageenan, alginates, karaya gums, xanthan gums, guar gums, gelatins, algins, tragacanth, chitosan, acrylamide polymers, carboxypolymethylenes, polyamines, poly quaternary compounds, polyvinylpyrrolidone, AVE/MA, salts of AVE/MA, mixed salts of AVE/MA, polymeric acids, polymeric salts, polyhydroxy compounds, and combinations thereof.

In one embodiment, the adhesive component is a salt of a polymer of AVE/MA. In another embodiment the adhesive component comprises a mixed salt of a polymer of AVE/MA. In a further embodiment, the AVE/MA copolymer contains a cationic salt function comprising a cation selected from the group consisting of: Group IA and Group IIA cations of the periodic table, yttrium, titanium, zirconium, vanadium, chromium, manganese, iron, nickel, copper, zinc, boron, aluminum, and combinations thereof. In another embodiment, the adhesive component is a mixed salt of an AVE/MA copolymer containing a cationic salt function comprising a cation selected from the group consisting of strontium, zinc, iron, boron, aluminum, vanadium, chromium, manganese, nickel, copper, yttrium, titanium, magnesium, calcium, sodium, and combinations thereof. In yet another embodiment the cation is selected from the group consisting of strontium, zinc, iron, magnesium, calcium, sodium, and combinations thereof. In one embodiment, the adhesive component comprises a calcium and zinc mixed salt of an AVE/MA copolymer. In another embodiment, the denture adhesive component comprises AVE/MA, salts of AVE/MA, mixed salts of AVE/MA, sodium carboxymethylcellulose, or combinations thereof. In another embodiment, the denture adhesive component is a combination of a mixed salt of AVE/MA and carboxymethylcellulose.

In further embodiments, the denture adhesive article comprises an additional adhesive component. In one embodiment, the additional adhesive component is present at the same levels and is selected from those listed for the adhesive component. In one embodiment, the additional adhesive component comprises a cellulose derivative. In a further embodiment, the cellulose derivative comprises sodium carboxymethylcellulose. In multiple embodiments, the additional adhesive component is present from about 5, 10, 15, 20% to about 30, 35, 40, 45, 50, 60%, or any combination thereof.

In one embodiment the denture adhesive component is not thermoplastic and/or comprises only low levels of water-soluble thermoplastic polymers, from about 0.01% to about 5% of water soluble thermoplastic polymers such as polyethylene oxide, hydroxypropyl cellulose, hydroxypropylmethylcellulose, and or polyethylene glycol; in another embodiment from about 0.01% to about 1% of water soluble thermoplastic polymer, or is essentially free of water soluble thermoplastic polymers.

In some embodiments when the article comprises three layers and has a denture adhesive component selected from the group consisting of sodium carboxymethylcellulose, polyethylene oxide, polyvinylalcohol, and mixtures thereof, then at least one outside layer comprises a water insoluble thermoplastic component and/or a viscosity index improver; and/or at least one layer comprises an additional denture adhesive component selected from the group comprising AVE/MA, salts of AVE/MA, mixed salts of AVE/MA, and mixtures thereof.

### Alkyl Vinyl Ether-Maleic Copolymer

In one embodiment of the invention the denture adhesive component is AVE/MA or salts of AVE/MA. The alkyl vinyl ether-maleic acid co-polymer comprises or consists essentially of the repeated structural unit:

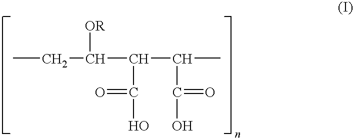

wherein R represents an alkyl radical, in one embodiment a C₁ to C₅ alkyl radical, n is an integer greater than one representing the number of repeated occurrences of the structural unit in a molecule of the polymer.

In one embodiment, the adhesive component is AVE/MA and salts thereof, preferably mixed salts of AVE/MA, wherein the copolymer contains a cationic salt function comprising a cation selected from the group consisting of Group IA and Group 2A cations of the periodic table, yttrium, titanium, zirconium, vanadium, chromium, manganese, iron, nickel, copper, zinc, boron, aluminum, and mixtures thereof. In another embodiment, the adhesive component is a mixed salt of AVE/MA containing a cationic salt function comprising a cation selected from the group consisting of strontium, zinc, iron, boron, aluminum, vanadium, chromium, manganese, nickel, copper, yttrium, titanium, magnesium, calcium, sodium, and mixtures thereof, and in yet another embodiment the cation is selected from the group consisting of strontium, zinc, iron, magnesium, calcium, sodium, and mixtures thereof.

AVE/MA contains, in one embodiment, a cationic salt function comprising from about 5% to about 50%, in another embodiment, from about 10% to about 40%, in yet another embodiment, from about 10% to about 35% (of the total initial carboxyl groups reacted) zinc cations. These zinc cations can be mixed with other cations selected from the group consisting of: from about 5% to about 65%, preferably from about 10% to about 60%, strontium cations, from about 0.001% to about 2.5%, preferably from about 0.01% to about 2% of iron, boron, aluminum, vanadium, chromium, manganese, nickel, copper, yttrium, and/or titanium cations, from about 5% to about 65%, preferably from about 15% to about 50% of calcium and/or magnesium cations and/or sodium cations.

AVE/MA and salts thereof, are also described in U.S. Pat. Nos. 5,073,604 to Holeva et al., issued Dec. 17, 1991; 5,525,652, issued Jun. 11, 1996, Clarke et al.; 6,025,411, issued Feb. 15, 2000, Wong et. al.; 4,758,630, issued Jul. 19, 1988, Shah et al.; 5,304,616, issued Apr. 19, 1994, Rajaiah et al.; 5,424,058, issued Jun. 13, 1995, Rajaiah; 5,424,058, issued Jun. 13, 1995, Rajaiah et al.; 4,758,630, issued Jul. 19, 1988, Shah et al.; 5,830,933, issued Nov. 3, 1998, Synodis et al.; 2,047,398, issued Jul. 14, 1936, Voss et al.; 3,003,988, issued Oct. 10, 1961, Germann et al.; 5,880,172, Rajaiah et al., issued Mar. 9, 1999; 5,900,470, Prosise et al., issued May 4, 1999; 5,037,924, Tazi et al., issued Aug. 6, 1991; 5,082,913, Tazi et al, issued Jan. 21, 1992; all of which are incorporated herein by reference in their entirety. Salts of AVE/MA are also described in P&G copending applications U.S. Pat. Nos. 6,355,706 to Rajaiah, et al., issued Mar. 12, 2002; 6,617,374 to Rajaiah, et al., issued Sep. 9, 2003.

In one embodiment the free acid level of the salts of the AVE/MA or AVE/MA/IB is at least about 36%, in another embodiment is from about 36% to about 60%, and even in another embodiment is from about 40% to about 55%, of the total initial carboxyl groups of the copolymer.

In one embodiment the specific viscosity of the starting copolymer acid or copolymer anhydride is from about 1.2 to about 14, when preferably measured in a 1% weight/volume solution in MEK (methyl ethyl ketone) at 25° C. Other methods and solvents can be used to measure the specific viscosity such as a 1% weight/volume solution in DMF (dimethyl formamide) at 25° C. and a 1% weight/volume solution in 2-butanone at 25° C.

Suitable AVE/MA copolymers may be prepared by well-known methods of the prior art; see, for example, U.S. Pat. No. 2,782,182, and U.S. Pat. No. 2,047,398.

Methods of making mixed salts of AVE/MA polymers are further disclosed in U.S. Pat. Nos. 5,073,604, Holeva et al., issued Dec. 17, 1991 and 5,872,161, Liang et al., issued Feb. 16, 1999.

### Water Insoluble Component

In some embodiments, the present article comprises a safe and effective amount of a water insoluble component. In one embodiment this component is present by weight of the article at an amount from about 2, 5, 10, 20, 25, 30, 35% to about 45, 50, 60, 70, 90%, or any combination thereof. In additional embodiments the water insoluble component is present at an amount from about 20% to about 70%, from about 25% to about 60%, or from about 35% to about 60% by weight of the article. In yet another embodiment the water insoluble component is substantially non-swellable in water. In some embodiments, the non-swellable water insoluble component swells less than about 10%, 5%, 2%, or 1% in water.

In one embodiment, the water insoluble component is selected from the group consisting of: natural wax, synthetic wax, petrolatum, polyvinyl acetate, natural oils, synthetic oils, fats, silicone, silicone derivatives, dimethicone, silicone resins, hydrocarbons, hydrocarbon derivatives, essential oils, caprilic/capric triglycerides, polybutene, oleic acid, stearic acid, and combinations thereof. In a further embodiment, the water insoluble component comprises petrolatum, polyvinyl acetate, natural oils, synthetic oils, fats, silicone, silicone derivatives, dimethicone, silicone resins, hydrocarbons, hydrocarbon derivatives, polybutene, oleic acid, stearic acid, essential oils, caprilic/capric triglycerides, or combinations thereof.

Examples of natural oils include, but are not limited to, vegetable oils (ex. corn oil), soy bean oils, cottonseed oils, palm oils, coconut oils, mineral oils, animal oils (ex. fish oils), etc. Examples of synthetic oils include, but are not limited to, silicone oils, etc. In one embodiment, the water insoluble component comprises a natural oil. In a further embodiment, the natural oil comprises mineral oil. In one embodiment, mineral oil is present in the article at an amount from about 30% to about 50% and in another embodiment, from about 35% to about 45%. In an additional embodiment, the water insoluble component is substantially free of petrolatum. In another embodiment, the water insoluble component further comprises petrolatum.

In some embodiments, the water-insoluble component is a wax. Waxes are generally made up of various substances including hydrocarbons (normal or branched alkanes and alkenes), ketones, diketones, primary and secondary alcohols, aldehydes, sterol esters, alkanoic acids, terpenes (squalene) and monoesters (wax esters). Different types of waxes include animal and insect waxes (beeswax, Chinese wax, shellac wax, spermaceti, lanolin), vegetable waxes (bayberry wax, candelilla wax, carnauba wax, castor wax, esparto wax, Japan wax, jojoba oil, ouricury wax, rice bran wax), mineral waxes (cresin waxes, montan wax, ozocerite, peat waxes), petroleum waxes (paraffin wax), and synthetic waxes (polyethylene waxes, Fischer-Tropsch waxes, chemically modified waxes, substituted amide waxes, polymerized α-olefins).

In one embodiment the water insoluble component is a natural or synthetic wax. In a further embodiment, the natural wax is selected from the group consisting of: animal wax, vegetable wax, mineral wax, and combinations thereof. In another embodiment, the animal wax includes beeswax, lanolin, shellac wax, Chinese wax, and combinations thereof. In another embodiment, the vegetable waxes include carnauba, candelilla, bayberry, sugar cane, and combinations thereof; and mineral waxes include fossil or earth waxes (ozocerite, ceresin, montan), and petroleum waxes such as paraffin, and combinations thereof. In one embodiment the waxes herein are natural waxes selected from the group consisting of beeswax, candelilla, candela, carnauba, paraffin, and combinations thereof. In varying embodiments, wax is present in an amount from about 1, 2, 5, 8% to about 10, 20%, or any combination thereof.

### Viscosity Index Improvers

As discussed previously, viscosity index improvers make the viscosity of denture adhesives more stable over a range of functional temperatures (i.e. about 25° C. to about 60° C.). It is believed that another mechanism also contributes to the improved properties of denture adhesives comprising viscosity index improvers. Without being limited by theory, it is believed at least some improved properties arise when at least some of the particles of an adhesive component are at least partially coated or surrounded by a viscosity index improver. In fact, it has been surprisingly discovered that a viscosity index improver, microcrystalline wax for example, can at least partially coat the particles of an adhesive component. This is especially seen when the denture adhesive article is made by heating up to or beyond the softening point of the viscosity index improver and then cooled to room temperature. In some embodiments, the viscosity index improver can coat the particles of the adhesive component by solidifying or crystallizing within the pores and/or crevices of particles of the adhesive component.

In some instances, the coating/surrounding of the adhesive component by the viscosity index improver functions as a physical barrier to protect the adhesive particles, for example, from being washed out due to incomplete hydration, excess hydration (from saliva or drinks), change in mouth temperature (ex. due to drinking a hot beverage like coffee), and/or chewing. This can also lead to a better utilization and optimization of the adhesive component which leads to a better performance. The increase in performance can lead to the ability to use less of the product to get the same or better hold as previous products.

Denture adhesives comprising a viscosity index improver tend to have a viscosity that is higher at elevated temperatures than those same compositions without a viscosity index improver. This is important because the denture adhesive is placed (along with the denture) into the mouth of a user which has a temperature generally higher than that of room temperature. Additionally, the temperature of a user's mouth can also be increased when ingesting hot beverages. The ability to maintain a higher viscosity at these higher temperatures contributes to better hold and less loss of the denture adhesive composition during use.

One way to determine whether a material would work as a viscosity index improver in a denture adhesive article of the present invention is to look at the instant viscosity ratio. The instant viscosity ratio is the ratio of the viscosities at room temperature (25° C.) and at an elevated temperature (40° C.) for a particular material.

The instant viscosity ratio can be measured as outlined further below. In one embodiment, the instant viscosity ratio of a material comprising a viscosity index improver is greater than about 0.25. In another embodiment, the instant viscosity ratio is from about 0.25 to about 1.0. In additional embodiments, the instant viscosity ratio is from about 0.25, 0.3, 0.4, 0.6, 0.7 to about 0.3, 0.4, 0.5, 0.8, 1.0, or any combination thereof. In a further embodiment, the instant viscosity ratio is from about 0.3 to about 0.8. In other embodiments, the instant viscosity ratio is from about 0.3 to about 0.6 or from about 0.3 to about 0.5.

The following describes two samples, a reference sample (RS) and a prototype sample (PS). The reference sample is considered the standard and is made using the standard water insoluble components, which would not include a viscosity index improver, while the prototype sample is made using a viscosity index improver. A general formula is given for a RS and a PS, then formulas of a specific RS and a specific PS are given, along with their instant viscosity ratios for comparison.

Procedure to Prepare a Reference Sample (RS) and a Prototype Sample (PS)

### Materials

1. Standard Denture Adhesive Components and Excipient Powders (to prepare samples of both the RS and PS):
   i. Ca(47.5)/Zn(17.5) MVE/MA (Methyl Vinyl Ether/Maleic Acid) mixed partial salt (33%)
   ii. Sodium Carboxymethylcellulose (20%)
   iii. Colloidal Silicon Dioxide (1.14%)
2. Water Insoluble Components (WIC) and Viscosity Index Improver
   iv. To prepare a sample of the RS using standard WIC:
      Mineral Oil (Drakeol 35 from Penreco, or Kaydol from Sonneborn) (23.95%)+White Petrolatum (“Snow” from Penreco) (21.91%)
      OR
   To prepare a sample of the PS using the prototype viscosity index improver and WIC:
      Mineral Oil (Drakeol 35 from Penreco, or Kaydol from Sonneborn) (40.812%)+Prototype viscosity index improver (5.048%)

### Procedure

The Reference Sample and Prototype Sample are both prepared using the following procedure:

Connect a mixer with wall-scraper blades (Unimix from Haagen and Rinau) and hot water jacket to a water bath and a vacuum pump. Set the water bath of the hot water jacket to about 95° C. Add the WIC and/or viscosity index improver ingredients to mixer vessel. If the water insoluble component and/or viscosity index improver are not liquid at room temperature, allow them to soften before turning on the agitator. Turn on the agitator to about 60 RPM; mix the WIC and/or viscosity index improver ingredient(s) until their temperature reaches about 95° C. Add the “Standard Denture Adhesive Components and Excipient Powders” via a funnel to the mixer with the vent open. Close the vent and stop mixing. Scrape off powder clumps. Re-start mixing at about 60 RPM. Pull about 24 inches Hg vacuum and mix until the batch reaches about 90° C. Reduce bath temperature to about 60° C. and continue mixing under vacuum until the batch reaches about 65° C. Stop mixing, turn off the pump, slowly open the vent, release the vacuum, and raise the lid. Fill the sample into a suitable container, such as a foil tube of about 1.4 oz in capacity. Allow samples to equilibrate for about one week. Just prior to testing, squeeze out and discard approximately the first 2 grams from the tube(s).

Whenever possible, the RS and PS are made with the same denture adhesive components and excipient powders at the same levels and with the same manufacturing procedure. This is done to provide a standard matrix to test the differences between a variety of viscosity index improvers by keeping all other variables including the denture adhesive components and sample preparation procedure the same. Among other properties imparted by the standard denture adhesive components, they also provide a standard driving force for the saliva and moisture to penetrate through the denture adhesive composition, and also provide a standard matrix to test the effect of a variety of viscosity index improvers.

If it is necessary to accommodate any property of the Prototype viscosity index improver or viscosity index improver/water insoluble component combination that is not accommodated by the process detailed above (for example if it softens only at temperatures greater than 95° C.), the processing temperature profile can be modified as needed. Similarly, if the above blend of standard denture adhesive components is not suitable, then, just a single denture adhesive component, for example, sodium carboxymethylcellulose at 53%, can be used instead of the blend with Ca/Zn MVE/MA salt. Additionally, if the above testing formulation gives a PS which is too thick to test for the instant viscosity ratio as described below, then the sample may need to be diluted with additional water insoluble component like mineral oil.

The above process tests for viscosity index improvers at a level of about 5%. It is believed that testing the prototype viscosity index improvers at 5% will help set-up a baseline, meaning that a finding of viscosity index improver properties at a level of 5% is indicative of viscosity index improver properties at high levels. That being said, a prototype viscosity index improver which is tested at 5% and is found not to have viscosity index improver properties at that level may have them at a higher percentage and should be tested at a higher level to confirm.

The above process can also be scaled up and used for general manufacturing at the temperature appropriate for the viscosity index improver and/or water insoluble component of the denture adhesive article.

The following table, Table 1, includes formulas for a particular reference sample (RS) and a particular prototype sample (PS). These are not examples of embodiments of the present invention, but are disclosed to illustrate the difference in instant viscosity ratio when a viscosity index improver is used, in this case, microcrystalline wax.

TABLE I

| | A RS | B PS |
|---|---|---|
| | % | % |
| Ca/Zn AVE/MA Salt | 33 | 33 |
| CMC | 20 | 20 |
| Silica | 1.14 | 0.00 |
| Mineral Oil | 23.95 | 40.812 |
| Petrolatum | 21.91 | 0.00 |
| Colloidal Silicon Dioxide NF | 0.00 | 1.14 |
| Microcrystalline Wax W-835 (by | 0.00 | 5.048 |
| Witco Crompton, Sonneborn) | | |

The instant viscosity of the PS at 25° C. is 211.4 Ps and at 40° C. is 80.7 Ps. This gives an instant viscosity ratio for the PS of 0.38. In contrast to this, the RS, made using the combination of mineral oil and petrolatum used in traditional denture adhesive creams, has an instant viscosity at 25° C. of 289.9 Ps and at 40° C. of 51.4 Ps. This gives an instant viscosity ratio for RS of 0.18. The higher instant viscosity ratio of PS shows that it is more temperature resistant than the reference/traditional water insoluble component and thus, microcrystalline wax will work as a viscosity index improver in that denture adhesive article.

Some examples of viscosity index improvers include polymethacrylates, olefin copolymers, hydrogenated styrene-diene copolymers, styrene polyesters, rubber, polyvinylchloride, nylon, fluorocarbon, polyurethane prepolymer, polyethylene, polystyrene, polypropylene, cellulosic resins, acrylic resins, microcrystalline wax, elastomers, poly(n-butyl vinyl ether), poly(styrene-co-maleic anhydride), poly(alkyl fumarate co-vinyl acetate), alkylated polystyrene, poly(t-butyl styrene), or combination thereof.

Examples of polymethacrylates include, for example, polyacrylate-co-methacrylate, polymethacrylate-co-styrene, or combinations thereof. Examples of elastomers include, for example, hydrogenated styrene-co-butadiene, hydrogenated styrene-co-isoprene, ethylene-ethylene-propylene polymer, ethylene-propylene polymer, styrene-ethylene-ethylene-propylene-styrene polymer or combinations thereof. An example of a rubber includes hydrogenated polyisoprene. Other examples of viscosity index improvers can be found in “Chemistry and Technology of Lubricants,” Chapman and Hall (2^{nd} Ed. 1997).

In another embodiment, the viscosity index improver is polyethylene, such as A-C 1702 or A-C 6702 made by Honeywell, with a penetration value of about 98.5 and about 90.0, respectively, under ASTM D-1321. In another embodiment, the viscosity index improver is substantially free of amorphous polyethylene having a molecular weight of at least about 80,000. In an additional embodiment, when the viscosity index improver consists of a polyethylene having an average molecular weight of from about 1000 to about 21,000 then the adhesive component is substantially free of a mixed partial salt of a lower alkyl vinyl ether-maleic anhydride salt of calcium and alkali cations selected from the group consisting of sodium, potassium, and quaternary ammonium cations.

In one embodiment the viscosity index improver is water-insoluble, hydrophobic, and/or thermoplastic.

In another embodiment, the viscosity index improver comprises microcrystalline wax. In one embodiment, the microcrystalline wax is refined and/or substantially pure. In an additional embodiment, petrolatum does not contribute the microcrystalline wax. In another embodiment, the microcrystalline wax has a melting point ranging from about 50° C. to about 100° C. In further embodiments, the microcrystalline wax has a melting point ranging from about 50° C., 55° C., 60° C., 65° C., 70° C. to about 70° C., 75° C., 80° C., 85° C., 90° C., 95° C., 100° C., or any combination thereof. In one particular embodiment, the microcrystalline wax has a melting point ranging from about 75° C. to about 85° C. In another embodiment the microcrystalline wax is manufactured by Crompton, Sonneborn (Witco) and referred to and sold under the trademark Mutiwax®W-835. In one embodiment, the viscosity index improver comprises microcrystalline wax and is present at an amount from about 10% to about 90% and in another embodiment from about 40% to about 60%.

In some embodiments, viscosity index improvers are used in an amount from about 1% to about 90%, and about 10% to about 90% in other embodiments. In another embodiment, the viscosity index improvers are used in an amount from about 40.0% to about 60.0%. In additional embodiments, the viscosity index improver is present from about 20, 30, 40, 45, 50, or 55% to about 25, 30, 40, 50, 60, 70, 80, or 90%, or any combination thereof. In one embodiment, the viscosity index improver is water soluble and/or non-swellable in water.

### Non-Adhesive Substrate

In one embodiment the present invention comprises at least one non-adhesive self supporting substrate. In one embodiment the non-adhesive self-supporting substrate is characterized by its ability to maintain strength and provide integrity for the article in the presence of water and/or saliva.

In one embodiment, the user or consumer is able to easily peel away the article in its original form, from the prosthesis after the article is used in the oral cavity as a denture adhesive.

In one embodiment the non-adhesive self-supporting substrate is a solid substrate material having a penetration value of less that about 20, in another embodiment less than about 10, and in another embodiment less than about 5, based on ASTM D1321 or ASTM D937. In one embodiment the non-adhesive self-supporting substrate is a solid substrate material having a penetration value of about 0.5, 1, 2 to about 10, 15, 18, 20, or any combination of these, based on ASTM D1321 or ASTM D937.

In one embodiment the non-adhesive self-supporting substrate is a substrate with a hardness value of greater than about 0.5, in another embodiment greater than about 1, and in another embodiment greater than about 2, under ASTM D5. In one embodiment the non-adhesive self-supporting substrate is a solid substrate material having a hardness value of about 0.5, 1, 2 to about 10, 15, 18, 20, or any combination of these, based on ASTM D5.

In one embodiment the non-adhesive self-supporting substrate may include materials such as polyester, polypropylene, nylon, rayon, cellulose acetate, non-adhesive cellulose derivatives, cellulose acetate, cloth, fibrous fleece, paper, plastic, leather, microcrystalline wax, synthetic fibers, natural fibers, and mixtures thereof. In one embodiment the non-adhesive substrate is selected from the group consisting of non-adhesive cellulose derivatives, polyester, polypropylene, nylon, rayon, cloth, paper, microcrystalline wax, and mixtures thereof. In another embodiment the non-adhesive substrate is selected from the group consisting of polyester, polypropylene, rayon, nylon, cloth, paper, and mixtures thereof; in another embodiment is polyester.

In one embodiment the non-adhesive self-supporting substrate may be in any physical form suitable for providing strength and/or integrity to the present denture adhesive component and/or water insoluble thermoplastic component. Such physical forms include extruded films and/or sheets, non-woven, woven, continuous, chopped, foam, and combinations thereof. In addition, the non-adhesive self-supporting layer may be formed by any process commonly known in the art. Such processes include extrusion, casting, calendaring, coating, un-bonded, spraybonded, spun-bonded, needle-punched, carded, thermal bonded hydroentangled, meltblown, aperture print bonded, needled, wet-laid, thy-laid, and combinations thereof.

In one embodiment the non-adhesive self supporting substrate has a flexural stiffness of less than about 10 grams/cm, in another embodiment less that about 5 grams/cm, in another embodiment less that about 3 grams/cm, in another embodiment less than about 2 grams/cm and in yet another embodiment from about 0.1, 0.5, 1, to about 2, 3, 5, 10 grams/cm, in any combination, flexural stiffness as measured on a Handle-O-Meter, model #211-300, available from Thwing-Albert Instrument Company of Philadelphia, Pa. as per test method ASTM D2923-95. Flexural stiffness is a material property that is a function of a combination of film/strip thickness, width and material modulus of elasticity. This test is a method for measuring the rigidity of polyolefin film and sheeting. It determines the resistance to flexure of a sample by using a strain gauge affixed to the end of a horizontal beam. The opposite end of the beam presses across a strip of the sample to force a portion of the strip into a vertical groove in a horizontal platform upon which the sample rests. A microammeter wired to the strain gauge is calibrated in terms of deflection force. The rigidity of the sample is read directly from the microammeter and expressed as grams per centimeter of the sample strip width.

### Miscellaneous Additives

### Plasticizing Agent

The articles of the present invention may also optionally comprise a safe and effective amount of one or more toxicologically-acceptable plasticizers. In varying embodiments the level of the plasticizing agent ranges from about 0.01% to about 40%, from about 1% to about 10%, or from about 2% to about 5% by weight of the article. In another embodiment the plasticizer is water insoluble.

Suitable plasticizing agents of the present invention include, but are not limited to, polyols (such as sorbitol); glycerin; propylene glycol; acetylated monoglyceride; hydrogenated starch hydrolysates; corn syrups; xylitol, glycerol monoesters with fatty acids; triacetin; diacetin; monoacetin; dimethyl phthalate; diethyl phthalate; dioctyl phthalate; diethylene glycol; triethylene glycol; tricresyl phosphate; dimethyl sebacate; ethyl glycolate; ethylphthalyl ethyl glycolate; o- and p-toluene ethyl sulfonamide; phthalic acid, glycerol triacetate, citric acid, phosphoric acid, glycol, a pentaerythritol ester of a fatty acid, stearic acid, glycerol monostearate, polyethylene glycol, butyl phthalyl butyl glycolate, dimethyl phthalate, dibutyl phthalate, triacetin, triethyl citrate, acetyl triethyl citrate, acetyl tributyl citrate, triphenyl phosphate, diethylene glycol, caprylic triglyceride, capric triglyceride, propylene glycol dicaprylate/caprate, their derivatives, or combinations thereof.

The articles of the present invention may also optionally comprise a safe and effective amount of one or more toxicologically-acceptable plasticizers. In varying embodiments the level of the plasticizing agent ranges from about 0.01% to about 40%, from about 1% to about 10%, or from about 2% to about 5% by weight of the article. In another embodiment the plasticizer is water insoluble.

Suitable plasticizing agents of the present invention include, but are not limited to, polyols (such as sorbitol); glycerin; propylene glycol; acetylated monoglyceride; hydrogenated starch hydrolysates; corn syrups; xylitol, glycerol monoesters with fatty acids; triacetin; diacetin; monoacetin; dimethyl phthalate; diethyl phthalate; dioctyl phthalate; diethylene glycol; triethylene glycol; tricresyl phosphate; dimethyl sebacate; ethyl glycolate; ethylphthalyl ethyl glycolate; o- and p-toluene ethyl sulfonamide; phthalic acid, glycerol triacetate, citric acid, phosphoric acid, glycol, a pentaerythritol ester of a fatty acid, stearic acid, glycerol monostearate, polyethylene glycol, butyl phthalyl butyl glycolate, dimethyl phthalate, dibutyl phthalate, triacetin, triethyl citrate, acetyl triethyl citrate, acetyl tributyl citrate, triphenyl phosphate, diethylene glycol, caprylic triglyceride, capric triglyceride, propylene glycol dicaprylate/caprate, their derivatives, or combinations thereof.

In another embodiment the plasticizer is water insoluble. In one embodiment the denture adhesive article, when extruded thermoplastically, does not cure and set as a result of the action of the plasticizer component. In another embodiment the plasticizer component does not solidify the viscosity index improver, the optional water insoluble component, and/or the denture adhesive article. Alternatively, in one embodiment the denture adhesive article may be substantially free of plasticizers.

### Gellant Agents

The articles of the present invention may also optionally comprise a safe and effective amount of one or more toxicologically-acceptable gellants. In varying embodiments, the level of the gellant agent ranges from about 0.01% to about 40%, from about 1% to about 10%, or from about 2% to about 5%, by weight of the article.

Suitable gellant agents of the present invention include, but are not limited to, polyvinylpyrrolidone/eicosene copolymer sold under the tradename Ganex® V-220F from ISP; tricontanyl polyvinylpyrrolidone sold under the tradename Ganex® WP-660 from ISP; and polyamide gellants including Sylvaclear®, Sylvacote®, Sylvagel®, and Uniclear® all available from Arizona Chemical; or combinations thereof.

### Therapeutic Actives

The denture adhesive articles may also comprise one or more therapeutic actives. Therapeutic actives may be present at a level of from about 1, 5, 10, 15, 20, 25, 30%, to about 3, 5, 10, 15, 20, 30, 50, 70%, or any combination thereof. Therapeutic actives include, for example, antimicrobial agents such as iodine, triclosan, peroxides, sulfonamides, bisbiguanides, or phenolics; antibiotics such as tetracycline, neomycin, kanamycin, metronidazole, cetylpyridinium chloride, domiphen bromide, or clindamycin; anti-inflammatory agents such as aspirin, acetaminophen, naproxen and its salts, ibuprofen, ketorolac, flurbiprofen, indomethacin, eugenol, or hydrocortisone; dentinal desensitizing agents such as potassium nitrate, strontium chloride or sodium fluoride; fluorides such as sodium fluoride, stannous fluoride, MFP (monofluorophosphate); anesthetic agents such as lidocaine or benzocaine; whitening agents such as peroxide; anti-fungals such as those for the treatment of candida albicans; insulin; steroids; herbal and other plant derived remedies; and baking soda. Other suitable therapeutic actives are discussed in the Physicians Desk Reference 62^{nd} Ed., 2008 and the Physicians Desk Reference for non-prescription drugs, dietary supplements, and herbs, 29^{th} Ed.

According to one embodiment, the active is selected from the group consisting of: anti-calculus, fluoride ion source, stannous ion source, whitening, antimicrobial, anti-plaque, anti-stain, anti-deposition, anti-gingivitis, anti-tartar, anti-periodontitis, anti-sensitivity, anti-cavity, anti-inflammatory, nutrients, antioxidants, anti-viral, anti-fungal, analgesic, anesthetic, H-2 antagonist, and combinations thereof.

### Other Miscellaneous Additives

Other suitable ingredients include colorants, preservatives (such as methyl and propyl parabens, for example), and rheology modifiers (such as silicon dioxide, for example). Rheology modifiers modify the rheological properties such as viscosity, elasticity, and or yield stress. The colorants, preservatives, and rheology modifiers may be present at levels of from about 0% to about 20%, by weight of the article, in another embodiment from about 0.1%, 0.2, 1, 2, 5, to about 1, 5, 10, 20%, or any combination thereof.

Additionally, the articles may also comprise one or more solvents. These optional solvents may be miscible with the viscosity index improver, water insoluble component, or both, and/or be capable of being dissipated in-situ. In one embodiment these solvents may be dissipated in-situ by evaporation, dissolution, dispersion, bio-absorption, or any other suitable means. In one embodiment, solvents include silicones, hydrocarbons, iso-dodecane, iso-hexadecane, iso-eicosane, polyisobutene, or combinations thereof.

### Flavors, Fragrance, Sensates

The articles of the present invention may also include one or more components which provide flavor, fragrance, and/or sensate benefit (warming or cooling agents). Suitable components include menthol, wintergreen oil, peppermint oil, spearmint oil, leaf alcohol, clove bud oil, anethole, methyl salicylate, eucalyptol, cassia, 1-8 menthyl acetate, sage, eugenol, parsley oil, oxanone, alpha-irisone, marjoram, lemon, orange, propenyl guaethol, cinnamon, vanillin, thymol, linalool, cinnamaldehyde glycerol acetal known as CGA, and mixtures thereof, as well as coolants. The coolant can be any of a wide variety of materials. Included among such materials are carboxamides, menthol, ketals, diols, and mixtures thereof. In one embodiment the coolants in the present articles are selected from the group consisting of the paramenthan carboxyamide agents such as N-ethyl-p-menthan-3-carboxamide, known commercially as “WS-3”, N,2,3-trimethyl-2-isopropylbutanamide, known as “WS-23,” and mixtures thereof. Additional preferred coolants are selected from the group consisting of menthol, 3-1-menthoxypropane-1,2-diol known as TK-10 manufactured by Takasago, menthone glycerol acetal known as MGA manufactured by Haarmann and Reimer, and menthyl lactate known as Frescolat® manufactured by Haarmann and Reimer. The terms menthol and menthyl as used herein include dextro- and levorotatory isomers of these compounds and racemic mixtures thereof. TK-10 is described in U.S. Pat. No. 4,459,425, Amano et al., issued Jul. 10, 1984. WS-3 and other agents are described in U.S. Pat. No. 4,136,163, Watson, et al., issued Jan. 23, 1979. These agents may be present at a level of from about 0% to about 40%, in another embodiment from about 0.05 to about 5%, and in another embodiment from about 0.1 to about 2%, by weight of the article.

### Backing Layer and Release Liner

The denture adhesive articles may be provided by itself or may be applied to, coated on, or otherwise provided with a backing layer. The backing layer can be provided as a single layer or as a laminate formed from a plurality of layers, such as any combination of a foam, mesh, and/or other suitable material. The backing layer can be water permeable, water impermeable, partially water permeable, water soluble, water insoluble, erodible, or a combination thereof. Additionally, the backing layer can be continuous or discontinuous (for example, formed from a plurality of discrete segments).

In one embodiment, the backing serves as a protective barrier for the adhesive and/or active. The barrier prevents substantial leaching and/or erosion of the adhesive and/or active by, for example, the wearer's lips, tongue, cheek, as well as saliva. This allows the denture adhesive article to adhere to the oral surface and/or denture for an extended period of time, from several minutes to several hours.

The backing may comprise polymers, natural and synthetic woven materials, non-woven material, foil, paper, rubber, and combinations thereof. The backing may be a single layer of material or a laminate of more than one layer. Preferably, the material is any type of polymer or combination of polymers that have flexural rigidity and are compatible with the denture adhesive articles. Suitable polymers include, but are not limited to, polyethylene, ethylvinylacetate, polyesters, ethylvinyl alcohol and combinations thereof. The backing is generally less than about 1 mm thick, preferably less than about 0.05 mm thick, and more preferably from about 0.001 to about 0.03 mm thick. A polyethylene backing is preferably less than about 0.1 mm thick and more preferably from about 0.005 to about 0.02 mm thick. The shape of the backing is any shape and size that covers the desired oral surface.

The backing is held in place on the oral and/or denture surface by adhesive attachment provided by the denture adhesive article. The general tackiness of the denture adhesive article causes the backing to be adhesively attached to the oral and/or denture surface without substantial slippage from the frictional forces created by the lips, teeth, tongue and other oral surfaces rubbing against the backing while talking, drinking, etc.

The denture adhesive articles may also further comprise a release liner. The release liner may be formed from any material which exhibits less affinity (including zero affinity) for the denture adhesive article than the denture adhesive article exhibits for itself and for the backing. In one embodiment, the release liner comprises a rigid sheet of material such as polyethylene, paper, polyester, or other material which is then coated with a non-stick type material.

### Process for Preparation of the Article

In some embodiments, the articles are formed by processes conventional in the arts, e.g. the film-making industries such as casting, coating, calendaring, extrusion. One example of a method for manufacturing includes: a) adding a viscosity index improver and/or water insoluble component to a vessel, b) heating and mixing the viscosity index improver and/or water insoluble component to at least about 55° C., and c) adding and mixing a denture adhesive component. The order of addition of the components is not believed to be critical so long as the adhesive component is present within the composition when the viscosity index improver and/or water insoluble component are substantially in liquid form. The temperature of the method will need to be adjusted based on the requirements for the viscosity index improver and/or water insoluble component being used. Thereafter, the melted mixture may be poured into molds or cast to an acceptable thickness on an appropriate substrate. Examples of such substrates include Mylar, continuous moving stainless steel belt (which may eventually enter a dryer section if needed), release paper, and the like. The articles are then cooled. The articles may then be dried if needed, e.g. in a forced-air oven. The temperature of the drying air and length of drying time depend on the nature of the solvent utilized, as is recognized in the art. Generally, the drying temperatures include a temperature between about 25° C. and 140° C., in another embodiment from about 60° and 90° C. for a duration of about 20 minutes to about 60 minutes, in another embodiment from about 30 minutes to about 40 minutes. The articles may then be cut into desired shapes with desired dimensions and then stacked and/or subsequently packaged. In one embodiment, after processing, the article is then die-cut into desired shapes. These shapes may facilitate application of the article to the dentures.

Another conventional film-making process known in the art is extrusion. This method is possible with films wherein the film-forming ingredient comprises a variety of extrudable materials. The mechanical particulars of the extrusion process, e.g. the particular equipment utilized, the extruding force, the shape and temperature of the orifice and/or dies, are considered to be within the skill of the art and can be varied in a known manner to achieve the physical characteristics of the articles described herein.

In one embodiment the thickness of the articles herein is generally between about 0.1 mm to about 2.5 mm, in another embodiment from about 0.4 mm to about 1.5 mm thick, and in another embodiment from about 0.5 mm to about 1 mm thick. The article may be thicker or thinner depending on the degree of cushioning desired by the user or wearer.

In one embodiment the articles herein may optionally be multiphase or have visually distinct phases. In another embodiment the articles herein may optionally have a release liner.

### Article Use

The present articles are generally applied to the denture prosthesis and thereafter the denture is secured to the oral cavity. In one embodiment the dentures are dried prior to application of the article. In one embodiment it is not necessary to wet the article and/or the denture prosthesis prior to applying it to the denture prosthesis in order to make the article stick to the denture prosthesis. The article may be applied to any suitable location on the prosthesis. In one embodiment the denture wearer generally wears the article from about 1 hour to about 3 days, in another embodiment from about 6 hours to about 24 hours. After usage the prosthesis is removed from the oral cavity, and any remaining part of the article may be cleaned from the prosthesis, for example by gentle scrubbing with water and a brush. In one embodiment the articles can be combined into a kit, and the kit can further comprise an indicia which indicates to the user not to wet the product prior to application to the denture or oral surface.

### Test Methods

To measure the instant viscosity ratio for a given material, for example RS or PS, one calculates the ratio of the material's viscosity at room temperature (25° C.) to its viscosity at an elevated temperature (40° C.), using the following procedure:

### Equipment:

Ares Strain-Controlled Rheometer

25 mm permanent parallel plates

### Method:

1. Load 25 mm parallel plates onto an Ares rheometer.
2. Zero the normal force.
3. Zero the gap @ 25° C. (i.e. room temperature).
4. Apply the sample of the material to the bottom plate in a semi circular motion moving across the plate. There should be enough specimen such that when a gap of 2.177±0.005 mm is reached and excess is trimmed, the specimen extends evenly to all edges of the plate with no gaps present.
5. Adjust the Gap using the following procedure:
   Click on set gap icon. Set command gap position to 2.55 mm.
   Set the Max Force Allowed to 100 g.
   Click on set Gap.
   Trim sample with plastic cover slide.
   Set the command gap position to 2.177 mm, Max Force Allowed=100 g.
   Click on set Gap.
   Trim sample with plastic cover slide.
   Set command gap position to 2.147 mm. Max Force Allowed=100 g.
   Click on set Gap.
   Do Not Trim Sample.
   Final Gap should read 2.147±0.005 mm
   Allow the temperature to equilibrate to 25° C.
   Record the Gap and the Axial Force in test notes along with any observations made.
   Start Experiment
6. Start test:
   Method is a Step Rate (Transient) test that runs the following procedure:
      i. Applies a rate of 0/s for 1 s (a 1 s delay)
      ii. Applies a rate of 5/s for 5 s
   Result should be a curve of Viscosity vs. Time
7. Record the peak viscosity (aka “Instant Viscosity”) of this curve.
8. Repeat steps 1-7 for the material at 25° C. —a minimum of three times
9. Repeat steps 1-7 for the material at 40° C. —a minimum of three times
10. Calculate the average value of the Instant Viscosity for the material at 25° C., and separately at 40° C.
11. Finally, calculate

“Instant Viscosity Ratio”=(Average Instant Viscosity for the material at 40° C.)/(Average Instant Viscosity for the material at 25° C.).

The bioerosion of the inventive articles can be measured by the following method: run a water source on top of the sample specimen for about 30 minutes while the specimen sits atop a wire mesh. The water source is a laboratory faucet adjusted such that the temperature is 39±1° C. and the flow rate is 16±1 ml/sec. Use a funnel to focus the flow and help dampen the effect of small pressure and temperature fluctuations within the water lines. The wire mesh grid has square openings approximately 0.09 inches×0.09 inches and is placed 2.5 inches below the tip of the funnel where it is clamped to a metal ring for support. Sample specimens weighing 0.025 g are placed on the mesh and images are taken at 0, 10 and 30 minutes to follow bio-erosion of the specimen. After 30 minutes the wire mesh containing the remainder of the specimen is removed and heated for 1 hour at 60° C. under vacuum to remove all remaining water. After the heating period, final weights are taken to calculate weight loss due to bio-erosion. An average of 3 specimens per sample are used to calculate bio-erosion time and weight loss. The article is bioerodible if it does not leave behind visible residue, film, or sheet after about 30 minutes under these testing conditions, and/or if it cannot be easily separated or peeled away manually in one or more large pieces after about 30 minutes under these testing conditions, and/or if it leaves behind less than about 2, less than about 4, less than about 6, and/or less than about 8% by weight of residue (of the original weight of the article) after about 30 minutes under these testing conditions. The above bio-erosion test may also be conducted at various time-points up to 8 hours.

The dry tack can be measured by the following method: 1. remove the article from the package material; 2. place the article on the palate-portion of a dry, acrylic upper-denture with the teeth facing downward; 3. apply pressure with fingers for about 3 to 10 seconds; 4. thereafter remove finger pressure; 5. then invert the denture with the teeth facing upward. In one embodiment the article demonstrates dry tack if: i. The article does not stick to fingers during steps 1-2, ii. leaves little or no residue on the fingers in steps 3-4, and iii. in step-5, the article does not fall off of the denture, once inverted, for at least about 10-30 seconds, or at least about 1 minute.

In another embodiment the article demonstrates dry tack if: i. The article does not stick to fingers during steps 1-4, and ii. in step-5, the article does not fall off of the denture, once inverted, for at least about 10-30 seconds, or at least about 1 minute.

In another embodiment the article demonstrates dry tack if in step-5, the article does not fall off of the denture, once inverted, for at least about 10-30 seconds, or at least about 1 minute.

The dry tack of the inventive articles can also be measured by the following procedure:

(a) Compress a 5 mm diameter disc (0.67 mm thick) sample of the article between a 1″ diameter cylindrical probe (made from polymethylmethacrylate) and a flat sheet of polymethylmethacrylate with a 2000 gram-force for 2 seconds; (b) Pull off the probe at 1 mm/second and record peak force; (c) Repeat procedure with no sample sandwiched between the two surfaces; and (d) Calculate: Dry Tack in grams/square centimeter=(Peak Force with Sample−Peak Force without sample)/Cross sectional area of sample disc.

In one embodiment, the above procedure is repeated with an applied force of 250 gram-force in step (a) and the tack measured in steps (b)-(d);

The article has dry tack if the tack measured with a 250 gram-force applied force is less than about 25, 50, 100, 200, or 500 grams/square-centimeter, and the tack measured with a 2000 gram-force applied force is greater than about 200, 500, 1000, 2000, 5000, 10000, or 25000 grams/square-centimeter, and any combination of these levels.

The loss modulus G″ and storage modulus G′ of the inventive articles can be measured by the following procedure:

(a) Load a sample disc of 8 mm diameter and 0.67 mm thickness onto a ARES rheometer using a parallel plate fixture with a compressive force of 500 grams; (b) Set strain to be 0.02%; (c) Measure G″ and G′ at a sweep of frequencies including 1 Hz.

The normalized dislodgement force and dislodgement force ratio of the inventive article can be measured by the following method:

Instrument: An Instron model 5544 is used. The load cell is calibrated according to manufacturer's specifications annually. The choice of load cell is determined by having the forces generated by the adhesive fall within the recommended operating range for the load cell. This is typically between 10%-90% of full capacity.

Test Fixtures: The geometry of a cylindrical probe and a flat plate are used as the test fixtures. The probe is made from PMMA, 0.2 sq.cm to 10 sq.cm in surface area. For the base plate, the same PMMA material is used but in sheet form, ¼″ thick. This is cut into 6″×6″ plates to be clamped onto the Instron.

Hydrating Liquid Artificial saliva containing low levels of various salts is used to hydrate the adhesive.

Artificial Saliva Composition

| Ingredient | | Amount per Liter | |
|---|---|---|---|
| K₂HPO₄ | | 4.2 | g |
| KH₂PO₄ | | 3.2 | g |
| KOH | | 2 | pellets |
| Mineral Stock Solution | | 5 | ml |
| KCl | 8 g per 100 ml of Stock Solution | | |
| NaCl | 8 g | | |
| Na2SO4 | 0.264 g | | |
| MgCl2•6H2O | 0.7687 (or 0.36 g Anhydrous MgCl2) | | |

Adhesive: 0.1 to 1.0 gram of adhesive is applied to the probe.

Hydration: The hydrating liquid (0.2 mL of artificial saliva to 2.0 ml) is pipetted onto the surface of the adhesive. The assembly is then permitted to hydrate for 20 minutes or more.

Test Method Once the sample is hydrated, it is mounted onto the Instron and the test is carried out via computer control. The method is comprised of the following steps:

(a) Compression to 750 to 7500 g of force; (b) Hold at compression for 2 minutes; (c) Reduce compressive force to 200 g_{f}; (d) Hold (1 minute); (e) Pull off at 1 mm/s; (f) Record Peak Dislodgement Force; (g) Calculate “Normalized Dislodgement Force”=(Peak Dislodgement Force)/(Surface Area of Probe); report in grams force per sq.cm; (h) Repeat steps (a)-(g) for commercial Fixodent Original denture adhesive (available commercially manufactured by P&G), or for the following reference formula: Ca(47.5%)/Zn(17.5%) MVE/MA salt 33%, sodium carboxymethylcellulose 20%, mineral oil USP (65-75 cst at 40 C) 23.93%, petrolatum USP (consistency 17-20 mm) 21.87%, colloidal silicon dioxide 1.14%, and Opatint OD1646 0.06%; suitable methods to make this reference formula are disclosed in U.S. Pat. No. 5,073,604, Holeva K., and U.S. Pat. No. 6,617,374 Rajaiah J.; (i) Calculate “Dislodgement Force Ratio”=(Peak Dislodgement Force of Prototype Adhesive)/(Peak Dislodgement Force of Fixodent Original or the reference formula described above.

Data: Each sample is repeated a minimum of 3 times and the average value of the “Normalized Dislodgement Force” and “Dislodgement Force Ratio” are reported.

Specifically the normalized dislodgement force and dislodgement force ratio can be measured by using the following parameters in the procedure: 0.25 gram adhesive; 1 inch diameter probe; hydration time of 20 minutes; and compression force of 7500 grams.

The “normalized ooze amount” and “ooze ratio” of the inventive article can be measured by the following procedure:

(a) Load initial sample weight of about 0.50 grams uniformly onto a 1 inch diameter cylindrical probe made from polymethylmethacrylate; (b) Bring probe to 1.2 mm of base plate, also made from polymethylmethacrylate; (c) Apply 750 gram force for 90 seconds; (d) At 90 seconds, trim and weigh material that has oozed out; (e) Calculate “Normalized Ooze Amount”=(Amount oozed out/Initial sample weight)×100; (f) Repeat Steps (a)-(e) using commercial Fixodent Original a denture adhesive cream commercially manufactured by P&G, or with the following reference formula: Ca(47.5%)/Zn(17.5%) MVE/MA salt 33%, sodium carboxymethylcellulose 20%, mineral oil USP (65-75 cst at 40 C) 23.93%, petrolatum USP (consistency 17-20 mm) 21.87%, colloidal silicon dioxide 1.14%, and Opatint OD1646 0.06%; (g) Calculate “Ooze Ratio”=Normalized Ooze Amount of Prototype Adhesive/Normalized Ooze Amount of Fixodent Original or the reference formula described above; (h) Each sample is repeated a minimum of 3 times and the average value of the “Normalized Ooze Amount” and “Ooze Ratio” are reported.

### EXAMPLES

The following examples further describe and demonstrate embodiments within the scope of the present invention. The examples are given solely for the purpose of illustration and are not to be construed as limitations of the present invention. Many variations of these are possible without departing from the spirit and scope of the invention.

### Example I

| | A | B | C | D | E | F | G | H |
|---|---|---|---|---|---|---|---|---|
| | % | % | % | % | % | % | % | % |
| Ca/Zn AVE/ | 33.00 | 33.00 | 33.00 | 53.00 | | 33.00 | 28 | 24.5 |
| MA Salt | | | | | | | | |
| CMC | 20.00 | 20.00 | 20.00 | | 53.00 | 25.00 | 15 | 28.5 |
| AVE/MA Acid | | | 1.00 | | | | | |
| S-97 | | | | | | | | |
| Micro- | 46.92 | 47.00 | 45.24 | 47.00 | 47.00 | 42.00 | 45.52 | 46.92 |
| crystalline | | | | | | | | |
| Wax¹ W-835 | | | | | | | | |
| Flavors | | | 0.50 | | | | 0.4 | |
| Sacharrin | 00.08 | | 0.16 | | | | 0.08 | 0.08 |
| Colorants | | | 0.10 | | | | | |
| Silica | | | | | | | 1 | |
| Corn Starch | | | | | | | 10 | |

¹Multiwax W 835 manufactured by Witco (Crompton, Sonneborn). The Multiwax W 835 may also be substituted with either Polyethylene A-C 1702 or Polyethylene A-C 6702.

The Microcrystalline Wax W-835 (or Polyethylene AC 1702 or A-C 6702) is melted, and the other ingredients are blended with it. The mixture is then made into sheets by any suitable means such as extrusion or rolling into sheets of suitable thickness such as 0.25 mm, 0.45 mm, 0.50 mm, 0.67 mm, 0.73 mm, or 1.0 mm. The sheet is then cut into shapes suitable for application to dentures.

### Example II

| | A | B | C | D |
|---|---|---|---|---|
| | % | % | % | % |
| Ca/Zn AVE/MA Salt | 33.00 | 33.00 | | 20.00 |
| CMC | 20.00 | 20.00 | 53.00 | 33.00 |
| Mineral Oil | 11.97 | 5.98 | 11.97 | 5.30 |
| Microcrystalline Wax² W-835 | 23.46 | 35.19 | 23.50 | 35.25 |
| Flavors | | | | 0.5 |
| Sacharrin | 0.04 | 0.06 | | 0.08 |
| Colorants | | | | 0.10 |
| Silica | 0.57 | 0.29 | 0.57 | 0.29 |
| Petrolatum | 10.96 | 5.48 | 10.96 | 5.48 |

²Multiwax W 835 manufactured by Witco (Crompton, Sonneborn). The Multiwax W 835 may also be substituted with either Polyethylene A-C 1702 or Polyethylene A-C 6702.

In Example II, the Microcrystalline Wax W-835 (or Polyethylene AC 1702 or A-C 6702) and Petrolatum are melted, and the other ingredients are blended with it. The mixture is then made into sheets by any suitable means such as extrusion or rolling into sheets of suitable thickness such as 0.25 mm, 0.45 mm, 0.50 mm, 0.67 mm, 0.73 mm, or 1.0 mm. The sheet is then cut into shapes suitable for application to dentures.

### Example III

| | A | B | C | D | E | F | G |
|---|---|---|---|---|---|---|---|
| | % | % | % | % | % | % | % |
| Ca/Zn AVE/MA Salt | | | | 33.00 | 33.00 | 30.00 | 10.00 |
| CMC | 10.00 | 20.00 | 30.00 | 20.00 | 20.00 | | |
| AVE/MA Acid S-97 | | | | 1.00 | | | |
| Microcrystalline | 90.00 | 80.00 | 70.00 | 30.00 | 23.50 | 70.00 | 90.00 |
| Wax³ W-180 | | | | | | | |
| Flavors | | | | 0.50 | | | |
| Saccharin | | | | 0.16 | | | |
| Colorants | | | | 0.10 | | | |
| Petrolatum | | | | 15.24 | 23.50 | | |

³Multiwax W 180 manufactured by Witco (Crompton, Sonneborn).

The Microcrystalline Wax W-180 is melted, and the other ingredients are blended with it. The mixture is then made into sheets by any suitable means such as extrusion or rolling into sheets of suitable thickness such as 0.50 mm, 0.67 mm, 0.73 mm, or 1.0 mm. The sheet is then cut into shapes suitable for application to dentures.

In the above examples, all or part of the Ca/Zn AVE/MA salt may be substituted with Mg/Zn/Na AVE/MA salts and/or Ca/Na AVE/MA salts; all or part of the CMC may be substituted with HEC, Caraggeenan, and/or Karaya Gum; all or part of the Microcystalline Wax W-180 may be substituted with Microcrystalline Wax W-445 (also available from Witco); all or part of the Petrolatum may be substituted with Mineral Oil, and/or Polybutene; and/or the amount of each ingredient may also be increased or decreased by up to about 50%. Each of the above examples may be blended with each other prior to making into sheets; and/or used in conjunction with each other to form multilayer articles.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A denture adhesive article, comprising:
(a) a denture adhesive component, and
(b) a viscosity index improver,
wherein the denture adhesive article is preformed.

2. The denture adhesive article of claim 1, wherein said viscosity index improver is selected from the group consisting of polymethacrylates, olefin copolymers, hydrogenated styrene-diene copolymers, styrene polyesters, rubber, polyvinylchloride, nylon, fluorocarbon, polyurethane prepolymer, polyethylene, polystyrene, polypropylene, cellulosic resins, acrylic resins, microcrystalline wax, elastomers, poly(n-butyl vinyl ether), poly(styrene-co-maleic anhydride), poly(alkyl fumarate co-vinyl acetate), alkylated polystyrene, poly(t-butyl styrene), and combinations thereof.

3. The denture adhesive article of claim 2, wherein the viscosity index improver is in an amount from about 10.0% to about 90.0% by weight of the denture adhesive article.

4. The denture adhesive article of claim 1, wherein the viscosity index improver is selected from the group consisting of microcrystalline wax, polyethylene, rubber, elastomers, and mixtures thereof.

5. The denture adhesive article of claim 1, wherein the viscosity index improver is selected from the group consisting of microcrystalline wax, polyethylene, and mixtures thereof.

6. The denture adhesive article of claim 4, wherein the viscosity index improver is in an amount from about 40.0% to about 60.0% by weight of the denture adhesive article.

7. The denture adhesive article of claim 1, wherein the denture adhesive component is selected from the group consisting of cellulose, cellulose derivatives, starch, starch derivatives, saccharide, saccharide derivatives, polyethylene oxides, polyethylene glycols, polyvinyl alcohols, carrageenan, alginates, karaya gum, xanthan gum, guar gum, gelatin, algin, tragacanth, chitosan, acrylamide polymers, carboxypolymethylene, polyamines, polyquaternary compounds, polyvinylpyrrolidone, AVE/MA, salts of AVE/MA, mixed salts of AVE/MA, polymeric acids, polymeric salts, polyhydroxy compounds, and combinations thereof.

8. The denture adhesive article of claim 7, wherein the denture adhesive component is in an amount from about 10.0% to about 60.0% by weight of the denture adhesive article.

9. The denture adhesive article of claim 8, wherein the denture adhesive component is selected from the group consisting of AVE/MA, salts of AVE/MA, mixed salts of AVE/MA, sodium carboxymethylcellulose, and combinations thereof.

10. The denture adhesive article of claim 9, wherein the denture adhesive component is in an amount from about 20.0% to about 55.0% by weight of the denture adhesive article.

11. The denture adhesive article of claim 1, further comprising a water insoluble component, wherein the water insoluble component is selected from the group consisting of petrolatum, polyvinyl acetate, natural oils, synthetic oils, fats, silicone, silicone derivatives, dimethicone, silicone resins, hydrocarbons, hydrocarbon derivatives, polybutene, oleic acid, stearic acid, essential oils, caprilic/capric triglycerides, and combinations thereof.

12. The denture adhesive article of claim 1, wherein the denture adhesive article is flexible, nonaqueous, and/or bioerodible.

13. The denture adhesive article of claim 1, wherein the denture adhesive article comprises a homogeneous mixture of (a) and (b).

14. The denture adhesive article of claim 1, wherein the denture adhesive article has a single layer.

15. The denture adhesive article of claim 1, wherein the denture adhesive article has dry tack properties.

16. The denture adhesive article of claim 15, wherein the shear storage modulus G′ measured in dynes/cm² at a frequency of about 1 Hz at about 25° C. is from about 3×10⁶ to about 1×10⁹.

17. The denture adhesive article of claim 1, wherein said denture adhesive article further comprises at least one non-adhesive self-supporting substrate.

18. The denture adhesive article of claim 1, wherein said denture adhesive article has a normalized ooze amount of from about 0% to about 20%, or an ooze ratio of from about 0 to about 0.7.

19. A denture adhesive kit comprising:
(a) a plurality of preformed denture adhesive articles comprising a safe and effective amount of a denture adhesive component; and
(b) an outer package or inner dispensing package having an upper denture indicia representing the placement of one or more articles only in the grooves of the denture.

20. A denture adhesive article, comprising:
(a) from about 10.0% to about 60.0% of a denture adhesive component comprising AVE/MA, salts of AVE/MA, mixed salts of AVE/MA, or a combination thereof; and
(b) from about 10% to about 90% of a viscosity index improver comprising microcrystalline wax;
wherein the denture adhesive article is preformed.
